(19) United States
(12) Patent Application Publication
(10) Pub. No.: US 2019/0247502 A1
(43) Pub. Date: Aug. 15, 2019

(54) ULTRASOUND TRIGGERED RELEASE FROM METAL ORGANIC FRAMEWORK NANOCARRIERS

(71) Applicant: American University of Sharjah, Sharjah (AE)

(72) Inventors: Ghaleb Husseini, Sharjah (AE); Rana Sabouni, Sharjah (AE); Mihad Ibrahim, Sharjah (AE)

(21) Appl. No.: 16/275,057

(22) Filed: Feb. 13, 2019

Related U.S. Application Data
(60) Provisional application No. 62/629,932, filed on Feb. 13, 2018.

(51) Int. Cl. A61K 41/00; A61K 9/00; A61P 35/00; A61K 9/51; A61K 31/704; A61K 31/475; A61K 33/243; A61K 31/337; A61K 31/513; A61K 31/07

(52) U.S. Cl. CPC A61K 41/0028; A61K 9/0009; A61P 35/00; A61K 9/5123; A61K 9/5115; A61K 31/07; A61K 31/475; A61K 33/243; A61K 31/337; A61K 31/513; A61K 31/704

(57) ABSTRACT

Metal-organic frameworks for drug delivery systems, therapeutic compositions including the metal-organic frameworks, and method of treating cancer using the therapeutic compositions are disclosed. According to some embodiments, a method of treating cancer in a mammal may include: providing a therapeutic composition containing a nanocarrier with a drug-loaded metal organic framework; delivering the therapeutic composition to the mammal; allowing the nanocarrier to circulate throughout a circulatory system of the mammal for a time sufficient to allow aggregation of a therapeutic quantity of the nanocarrier at a treatment area; and applying ultrasound to the treatment area such that the nanocarrier releases the drug in the treatment area.

### CROSS-REFERENCE TO RELATED APPLICATIONS

This application claims priority to U.S. Provisional Patent Application No. 62/629,932, filed on Feb. 13, 2018, which is hereby incorporated by reference in its entirety and should be considered a part of this specification BACKGROUND

### FIELD

The present application relates generally to systems and methods for producing acoustically activated or triggered nanoparticles and more specifically, relates to systems and methods for producing and using acoustically activated or triggered, targeted metal organic frameworks (MOFs) for the novel treatment of cancer.

### DESCRIPTION OF THE RELATED ART

Cancer is a disease commonly understood to be caused by an abnormal, uncontrolled cell growth, which may invade neighboring and/or surrounding tissues and organs. Neoplasms and malignant tumors are other terms used to name cancer. Cancer can be grouped into at least the following categories: carcinoma, which includes cancers that start in the skin or tissues that cover organs; sarcoma, which includes cancers occurring in connective or supportive tissues (e.g., bone and muscles); leukemia, which includes cancers that originate in blood forming tissue; lymphoma and myeloma, which includes cancers affecting the cells of the immune system; and central nervous system cancers.

Cancer, which may be (and frequently is) a lethal disease, has been cited as the second most common cause of death in the USA. Fifteen million cancer cases are predicted to be diagnosed by the year 2020, and about twelve million cancer patients are expected to die. Table 1, below, provides select 2012 cancer statistics in the United States. As can be seen, the three most commonly diagnosed cancers among men are prostate, lung, and colorectal cancers, whereas, breast, lung, and colorectal cancers are the most commonly diagnosed types among women. In addition, men may have a higher overall probability of being diagnosed with cancer compared to women.

TABLE 1
Cancer Statistics, United States, 2012

| Comparison | Women | Men |
|---|---|---|
| Most Commonly | 1. Breast | 1. Breast |
| Diagnosed | 2. Lung and Bronchus | 2. Lung and Bronchus |
| Cancers | 3. Colorectal | 3. Colorectal |
| Lifetime Probability to | Lower | Higher |
| be Expected with Cancer | | |

Cancer treatments are frequently grouped into four categories, including: 1) surgery, 2) chemotherapy, 3) radiation, and 4) antibody blocking therapy (these four treatment schedules may be and frequently are combined in an effort to achieve an improved outcome). For example, more selective and effective cancer therapies may comprise a combination of chemotherapy, radiation, and antibody blocking therapy, to reduce the impact of chemotherapeutic drugs on healthy cells.

More recently, various drug delivery strategies have been employed with different types of drug carriers to provide effective delivery of a drug payload to target cancer cells. In particular, nanoparticles have been demonstrated to act as effective drug carriers, however, efficient nanoparticle release of the carried drug to the proper target cells remains a challenge. Therefore, there is a need for improved drug carriers for drug delivery systems used in cancer treatment.

### SUMMARY

In some embodiments, a method of treating cancer cells in a mammal is provided. The method comprises: providing a therapeutic composition, the therapeutic composition comprising a nanocarrier comprising a metal organic framework with therapeutic drug loaded therein, the metal organic framework configured to release the therapeutic drug upon exposure to ultrasound; delivering the therapeutic composition to the mammal; allowing the nanocarrier to circulate throughout a circulatory system of the mammal for a time sufficient to allow aggregation of a therapeutic quantity of the nanocarrier at a treatment area; and applying ultrasound to the treatment area such that the nanocarrier releases the drug in the treatment area.

In some embodiments, the metal organic framework may comprise iron (III) iron (Fe³⁺) and 2,6 naphthalenedicarboxylic acid. Particles of the metal organic framework may have a rod-like shape. Particles of the metal organic framework may have an average diameter between 50-100 nm and/or an average length between 200-600 nm. The ultrasound applied to the treatment area may comprise a low frequency ultrasound. The ultrasound applied to the treatment area may have a frequency between 20 kHz and 40 kHz. The ultrasound applied to the treatment area may have a power density of 0.1-2.0 W/cm². The ultrasound may be applied with an ultrasound probe. The ultrasound may be applied for 30 minutes or less. More than 20% of the loaded drug may be released from the metal organic framework after applying the ultrasound. The drug may be a chemotherapeutic drug. The drug may be selected from the group consisting of doxorubicin, annamycin, daunorubicin, vincristine, cisplatin derivatives, paclitaxel 5-fluorouracil derivatives, camptothecin derivatives, and retinoids.

In some embodiments, a therapeutic composition is provided. The therapeutic composition comprises a nanocarrier comprising a metal organic framework, wherein the metal organic framework comprises iron (III) iron (Fe³⁺) and 2,6 naphthalenedicarboxylic acid; and a drug loaded at the metal organic framework.

In some embodiments, particles of the metal organic framework may have a rod-like shape. Particles the metal organic framework have an average diameter between 50-100 nm and/or an average length between 200-600 nm.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 illustrates X-ray diffraction (XRD) results of Fe-NDC-M and Fe-NDC-O.

FIG. 2A is a scanning electron microscopy (SEM) image of Fe-NDC-M.

FIG. 2B is a scanning electron microscopy (SEM) image and Fe-NDC-O.

FIGS. 3A-3B illustrate Energy-dispersive X-ray (EDS) quantitative analysis results of Fe-NDC-M.

FIGS. 3C-3D illustrate Energy-dispersive X-ray (EDS) quantitative analysis results of Fe-NDC-O.

FIG. 4 illustrates FT-IR patterns of Fe-NDC-M and Fe-NDC-O.

FIG. 5A illustrates thermogravimetric analysis (TGA) results of Fe-NDC-M.

FIG. 5B illustrates thermogravimetric analysis (TGA) results of Fe-NDC-O.

FIG. 6A illustrates the BJH absorption cumulative pore volume of Fe-NDC-M.

FIG. 6B illustrates the BJH absorption cumulative pore volume of Fe-NDC-O.

FIG. 7A illustrates the release percentage of calcein salt from Fe-NDC-M over time with or without the application of 40 kHz ultrasound.

FIG. 7B illustrates the release percentage of calcein salt from Fe-NDC-O over time with or without the application of 40 kHz ultrasound.

FIG. 7C illustrates the release percentage of calcein salt from Fe-NDC-M and Fe-NDC-O over time without the application of ultrasound.

FIG. 7D illustrates the release percentage of calcein salt from Fe-NDC-M and Fe-NDC-O over time with the application of 40 kHz ultrasound.

FIG. 8A illustrates the release percentage of calcein salt from Fe-NDC-M over time with or without the application of 40 kHz ultrasound and with mixing.

FIG. 8B illustrates the release percentage of calcein salt from Fe-NDC-O over time with or without the application of 40 kHz ultrasound and with mixing.

FIG. 9 illustrates release percentage of calcein salt from Fe-NDC-M over time with or without the application of 20 kHz ultrasound.

FIG. 10 is a scanning electron microscopy (SEM) image of Fe-NDC-M.

FIG. 11 illustrates the absorbed amounts of gas measured as a function of relative pressure of Fe-NDC-M.

FIG. 12 illustrates the absorbance of calcein salt solution with various concentrations at wavelengths from 400 nm to 550 nm.

FIG. 13 illustrates the fluorescence intensity of calcein salt solution with difference concentrations.

FIG. 14 illustrates the absorbance of calcein salt solution with different concentrations.

FIG. 15 illustrates the absorbance of calcein salt solution and supernatants at wavelengths from 400 nm to 500 nm.

FIG. 16 illustrates the cumulative release % of calcein salt from Fe-NDC-M over time with or without application of 40 kHz ultrasound.

FIG. 17 illustrates a cumulative fraction of calcein salt released over time without application of ultrasound and line fitting.

FIG. 18 illustrates a cumulative fraction of calcein salt released over time with application of 40 kHz ultrasound and line fitting.

FIG. 19 illustrates the cell viability of MCF-7 cells after different concentrations of Fe-NDC-M solution was introduced.

### DETAILED DESCRIPTION

Nanotechnology may be used as a platform for many anticancer drug delivery systems (DDS). Encapsulating antineoplastic agents in nanocarriers may advantageously allow targeting, e.g., specific targeting, of cancerous cells or tissues to avoid potentially poor selectivity of conventional free chemotherapeutic drugs, e.g., free in a patient's circulatory system. Thus, nanocarriers may advantageously avoid one or more drawbacks commonly associated with conventional chemotherapeutic drugs, including, but not limited to, rapid resistance and clearance, undesired side effects on normal cells, and/or poor pharmacokinetics. Features that may be considered when designing and/or optimizing drug carriers, e.g., effective drug carriers, such as those disclosed herein, include, but are not necessarily limited to, size (e.g., small size), toxicity (e.g., non-toxicity), biocompatibility, drug entrapment (e.g., the ability to entrap high concentrations of a drug), controllability of release, and ability to be detected by imaging techniques.

Nanomaterials used for loading anticancer drugs may be divided into two broad categories: organic carriers (such as: liposomes, micelles, and dendrimers); and inorganic carriers (such as: mesoporous silica, carbon tubes, and zeolites). A third class/category of nanocarrier drug delivery system (e.g., anticancer drug carriers), metal organic frameworks (MOFs), may advantageously overcome certain limitations that may be experienced by organic and/or inorganic drug delivery systems. MOFs are self-assembling hybrid materials that may be synthesized by combining inorganic clusters (e.g., metal salts) with organic compounds (e.g., bridging linkers) via different preparation routes and/or methods to form network arrangements with generally well-defined crystallinity and generally high porosity. MOFs may be defined as hybrid (organic-inorganic) crystalline porous polymers.

The presence of organic and inorganic groups, regular and tunable porosity, high crystallinity, as well as the possibility of functionalization of MOFs with targeting moieties may contribute to the characteristics/properties/abilities of MOFs, including, but not limited to: compatibility, ability to uptake high payloads of drugs with controlled release, and the localized delivery of drugs, in the hope of increasing treatment efficiency, e.g., cancer treatment efficiency. MOFs may be used in any of a number of drug delivery systems in which it is desirable to selectively deliver a drug or other substance to a tissue or cell of interest—cancer is only one of many examples to which MOFs may be advantageously applied.

One MOF family comprising MIL (Materials of Institut Lavoisier) may be used as smart drug carriers. For example: drugs, e.g., ibuprofen, may be encapsulated in MIL-100 and/or MIL-53; drugs, e.g., busulfan, may be encapsulated in MIL-89, MIL-88A, MIL-100 and/or MIL-53; drugs, e.g., doxorubicin and/or topotecan, may be encapsulated in MIL-10 and/or others. Other MOFs may also be used as drug delivery systems. For example camptothecin, doxorubicin and/or 5-fluorouracil may be loaded in ZIF-8, mitroxantron and/or [Ru(p-cymene)Cl₂(pta)] (RAPTA-C) in ZnBDP_X series; and doxorubicin may be loaded in Gd-pDBI and/or MG-Gd-pDBI.

Targeting mechanisms for anticancer site-specific drug delivery systems based on nanovehicles include passive targeting and active targeting (also referred to as “ligand targeting”). These classifications are generally applied to therapeutic systems capable of accumulating at the treatment, e.g., diseased, site. Differences between the target-area microenvironment, e.g., cancer tissue microenvironment, and the microenvironment of normal tissues, e.g., in terms of morphology, pH, and/or other factors, may be used to develop/design/tune targeted nanoparticles-based therapies, e.g., antitumor therapies.

Tumors may have, e.g., generally have, microvessels with a vascular pore cutoff size ranging between about 380 and 780 nm. However, depending on the location, type, and environment of the tumor, e.g., cancer cells, the size of these fenestrations may reach 800-2000 nm in size. Passive targeting-based systems rely, at least in part (e.g., a significant part), on fenestrations found in defective and leaky blood microvessels and poor lymphatic drainage commonly found in tumors, which result in the enhanced permeability and retention (EPR) effect. The EPR effect permits large molecules, e.g., having a molecular weight greater than about 40-50 kDa (such as, but not limited to, proteins and polymers) to accumulate effectively and be retained by cancer tissues for a longer period of time than is generally observed in normal/healthy tissues. Myocet and Doxil (liposome containing doxorubicin) are examples of EPR-based passive targeting systems that have been used in humans.

Active targeting-based systems rely, at least in part (e.g., a significant part), on functionalization to improve selectivity. For example, nanocarriers may be advantageously functionalized by conjugating a ligand (e.g., a targeting moiety) to their surface for which the targeted cancer cells have over-expressed receptors. Ligand targeting generally occurs after (or during/as) the nanocarrier has passively accumulated the tissue of interest, e.g., in the tumor tissue; consequently, this mechanism may work particularly effectively in conjugation with passive targeting and the EPR effect. Ligands that may be conjugated to the surface of one or more active drug delivery systems (e.g., to be used for active targeting) may include peptides, antibodies and their fragments, and aptamers. Several moieties have been approved for clinical active targeting drug delivery systems, including, the anti-HER2 trastuzumab (Herceptin), the anti-EGFR cetuximab, rituximab (Rituxan). Such ligands may be conjugated on/to the surface of nanoparticles to increase binding affinity to receptors that may be overexpressed on the surface of cells, e.g., cancer cells.

One or more nanoparticle characteristics may be controlled or modulated to alter (e.g., increase or decrease) circulation time and or clearance rate. For example, intravenously injected formulations of encapsulated drugs, the size and shape of nanoparticles may be controlled to advantageously alter (e.g., prolong) circulation time and clearance (e.g., avoid rapid clearance) by the liver and/or kidneys. Clearance, e.g., rapid clearance, can also be modulated (e.g., decreased or avoided) by coating the surface of nanocarriers with polymers (to alter their hydrophilicity).

The shape of a nanocarrier may also play a role in the particle's internalization efficiency. In some embodiments, the shape of a nanocarrier may have a rod-like shape. Rod-like shaped carriers may have different internalization efficiency in several cancer cell lines (e.g., three different breast cancer cell lines (BT-474, SK-BR-3, and MDA-MB-231) than spherical carriers. Mesoporous silica nanoparticles (MSNP) having rod-like structures with dimensions of 160-190/60-90 nm may be taken up preferentially by HeLa cells compared to 110-nm diameter MSNP spheres. Poly-ethylene glycol (PEG) hydrogel rods with aspect ratio of 150×450 nm may experience a comparatively higher uptake than cylindrical particles of 200×200 nm. Trastuzumab-coated rods (367±33 nm in length and 126±8 nm in width) may experience a comparatively higher uptake than 200-nm spheres. As disclosed herein, Fe-NDC-MOFs nanorods may advantageously be used as anticancer drug carriers due to their rod shape, which may enhance the nanocarriers' cellular uptake by comparison to spherical MOFs.

Ultrasound (US) may be used as a modality to trigger release in/from various drug delivery systems, including but not limited to nanoemulsions, micelles, liposomes, and polymeric nanoparticles. This technique represents an avenue (e.g., an effective way) to enhance drug release (due, at least partially, to acoustic oscillation) from nanocarriers by local sonication (e.g., application of ultrasound at/to the treatment area (e.g., tumor site). Ultrasound may also advantageously enhance drug transport into the cytosol of cells, e.g., tumor cells, via sonoporation.

In embodiments, acoustically-activated drug delivery may rely on two or more potential pathways, including, but not necessarily limited to, thermal effects and non-thermal effects. The thermal effects of ultrasound include the interaction between ultrasound and biological tissues due to localized temperature increase in the tissues which occurs as a result of acoustic energy being absorbed when ultrasound, e.g., high intensity focused ultrasound (HIFU), is applied. The non-thermal effects of ultrasound are due, at least partially, to mechanical and cavitation effects. Mechanical effects may be caused by the motion of the fluid and the nanocarrier via pressure waves and acoustic streaming, both of which have the capability of increasing drug transport in the cells. Cavitation is the formation and oscillation of very small gas bubbles (microbubbles) in tissues due to ultrasound-induced vibrations. Cavitation can generate high stresses on the cell membranes and the nanocarrier, resulting in the possible collapse and release of the encapsulated drug.

### Synthesis of MOFs

In certain embodiments, MOFs may be synthesized from a metal salts/metal ions and organic ligands. In some embodiments, the organic ligands may be any suitable mono-, di-, tri-, or tetravalent ligands. The metal salt/metal ion may be of any suitable metal, such as a transition metal, for example: Iron (Fe), Titanium (Ti), or zirconium (Zr). In some embodiments, MOFs, including those based on Fe-NDC-M and Fe-NDC-O (described below), may be synthesized using iron nitrate nonahydrate (Fe(NO₃)₃.9H₂O), and 2,6 naphthalenedicarboxylic acid (2,6-NDC). In some embodiments, iron nitrate nonahydrate and 2,6-NDC may be reacted in the molar ratio of 10-1:10, 5:1-1:5, 3:1-1:3. 2:1-1:2, 1.5:1-1:1.5, or 1:1. Iron nitrate nonahydrate and 2,6 NDC may be reacted in a solvent. In some embodiments, the solvent may be dimethylformamide (DMF), dimethylacetamide (DMAC) or dimethylsulfoxide (DMSO). Iron nitrate nonahydrate and 2,6 NDC may be stirred in the solvent.

Then the mixture may be reacted. In some embodiments, the mixture may be subject to microwave irradiation. For example, the mixture may be heated in a microwave oven. The mixture may be subject to microwave irradiation of about 10 W-500 W, 10 W-300 W, 10 W-300 W, 20 W-250 W, 30 W-250 W, 50 W-250 W, 100 W-200 W, or 150 W-200 W. The mixture may be irradiated for 30 sec or longer, 1 min or longer, 2 min or longer, 3 min or longer, 5 min or longer, or 10 min or longer. As described herein this section or elsewhere in this specification, the MOFs produced from microwave irradiation may be referred as “Fe-NDC-M”.

In some embodiments, the mixture may be heated instead of or in addition to microwave irradiation. For example, the mixture may be heated in an oven, such as a conventional electrical oven. In some embodiments, the mixture may be heated at 50° C.-200° C., 50° C.-170° C., 70° C.-170° C., 70° C.-150° C., 70° C.-130° C., 80° C.-120° C., or 90° C.-110° C. In some embodiments, the mixture may be heated for 3 hours or longer, 5 hour or longer, 10 hours or longer, 15 hours or longer, 20 hours or longer, or 24 hours or longer. As described herein this section or elsewhere in this specification, the MOFs produced from heating may be referred as “Fe-NDC-O”.

After the reaction, the product MOF (Fe-NDC) may be produced. The product may be separated from the reaction mixture, for example by centrifuge, washing and/or drying.

In some embodiments, the resulting MOF particles may have a pale yellow color. In some embodiments, the MOF particles may have crystalline structures. In some embodiments, the MOF particles may have a rod-like shape. Rod-shaped nanoparticles may target several cancer cell lines effectively, as compared to other crystalline geometries, so the rod-like shape of MOF may contribute to effectiveness of the MOF as a nanocarrier.

The MOF particles may have a size in the nano-range. For example, in some embodiments, the MOF particles may have an average diameter at least substantially within the size range between about 10-200 nm, 20-150 nm, 30-130 nm, 30-120 nm, 40-110 nm, 50-100 nm, or 50-80 nm. In some embodiments, the MOF particles may have an average length at least substantially within the range of 50-900 nm, 70-800 nm, 100-700 nm, 200-600 nm, or 300-450 nm. In some embodiments, the MOF particles may include 55-65 wt % carbon (C), 35-45 wt % oxygen (O), and 0.1-2 wt % of iron (Fe).

The MOF particles may have pores. In some embodiments, the pores may have an average diameter of about 50-400 \acute{Å}, 50-350 \acute{Å}, 75-350 \acute{Å}, 75-300 \acute{Å}, 100-200 \acute{Å}, 120-160 \acute{Å}, or 130-150 \acute{Å}.

### Drug Loading

Drug may be loaded in synthesized MOFs, e.g., Fe-NDC-M and Fe-NDC-O MOFs, by stirring already-synthesized MOFs with a drug solution. In the drug solution, the drug may be dissolved in a solvent, such as neutral phosphate buffered saline (PBS). Drops of HCl/NaOH may be added to maintain the pH, for example pH 7-10, pH 7-9, or pH 7-8. In some embodiments, the MOFs and the drug solution may be stirred for 3 hours, 6 hours, 12 hours, a day, 2 days, 3 days, 1 week, 2 weeks, 1 month or more. The loaded MOF particles may be collected from the resulting mixture, for example by centrifuging, drying, and/or washing.

In embodiments, the loading mechanism of molecules within a MOF may be via adsorption of the guest molecules onto the interior surface or inside the framework pores. There are several forces that may govern this adsorbate-adsorbent interaction including: hydrogen bonds, hydrophobic (van der Waals) forces, coordination bonds, or π-π interactions.

Loading efficiency, the ratio of the amount of the drug adsorbed by MOFs to the amount of the drug in the drug solution (before loading) may be calculated. In some embodiments, the loading efficiency may be 20% or more, 30% or more, 40% or more, 50% or more, 70% or more 80% or more, 85% or more, 90% or more, 93% or more, 95% or more, 98% or more, or 99% or more. Loading capacity, the weight ratio of the drug loaded on MOFs to the total weight of the MOF and the drug loaded on MOF, may be also calculated. In some embodiments, the loading capacity may be may be more than 5%, more than 7.5%, more than 10%, more than 11%, more than 12%, more than 13%, more than 14%, more than 15%, more than 16%, more than 17%, more than 20%, or more than 30%.

The drug loaded to the MOFs may comprise calcein. In some embodiments, the chemotherapeutic drug may be selected from the group of doxorubicin, annamycin, daunorubicin, vincristine, cisplatin derivatives, paclitaxel 5-fluorouracil derivatives, camptothecin derivatives, and retinoids.

### Drug Release

A therapeutic composition including the drug-loaded MOFs may be administered to the patient. The therapeutic composition may further include any suitable additional carriers or pharmaceutically active or inactive constituents. The therapeutic composition may be administered to the body of a patient in various ways, including but not limited to: oral administration, intravenous injection, intramuscular injection, or subcutaneous injection.

When the drug loaded MOFs are administered to a patient, the drug loaded to the MOFs may be released. In some embodiments, at least some of the drug may be released from MOFs over certain time, naturally without any external intervention. In some embodiments, the ratio of the drug released from MOFs after 10 minute, may be 0.5% or less, 0.75% or less, 1% or less, 2% or less, 5% or less, or 10% or less of the loaded drug.

In some embodiments, an additional procedure may be conducted to promote or trigger release of the drug from the MOFs. For example, ultrasound may be applied to the treatment area to trigger release of the drug from the MOFs. In some embodiments, the ultrasound may be the low frequency ultrasound, and may have frequency in the range of 20-40 kHz, 20-30 kHz, or 30-40 kHz. In certain embodiments, the frequency may be below 20 kHz, or from 40-100 kHz, 100-200 kHz, or more than 200 kHz. In some embodiments, the ultrasound may have a power density of 0.1-2.0 W/cm², 0.1-1.5 W/cm², 0.1-1.3 W/cm², or 0.2-1.0 W/cm². The ultrasound may be applied for 10 seconds or less, 30 seconds or less, a minute or less two minutes or less, five minutes or less, ten minutes of less, or thirty minutes or less. In certain embodiments, ultrasound may be delivered for longer than 30 minutes. In some embodiments, when the ultrasound is applied, the ratio of the drug released from MOFs after 10 minute, may be 10% or more, 20% or more, 30% or more, 40% or more, 50% or more, 60% or more, 70% or more, 80% or more, or 90% or more. In some embodiments, a clinician may wait before applying the ultrasound to allow the MOFs to circulate throughout a circulatory system of the patient or subject for a time sufficient to allow aggregation of a therapeutic quantity of MOFs (and drug loaded on MOFs) at the treatment area.

MOFs may be advantageously used in various drug delivery systems that operate by injecting the loaded nanocarriers directly to or around a treatment site, e.g., a tumor site, and focusing ultrasound on the treatment site, e.g., the diseased location, to trigger release in the treatment site, e.g., in and to the cancer cells. Using MOFs to selectively target drug delivery may advantageously reduce potential adverse side effects of encapsulated chemotherapeutic drug on normal cells (compared to administering free drug). To use MOFs as or in intravenous formulations, they can be coated or functionalized using certain targeting moieties to avoid release while circulating in the blood.

### EXAMPLES

### Example 1-1: Synthesis of MOFs by Means of Microwave Irradiation

0.2307 mmol (93.2 mg) of iron (III) nitrate nonahydrate (Fe(NO₃)₃.9H₂O, ACS reagent, ≥98%) and 0.2307 mmol (49.9 mg) of 2,6 naphtalenedicarboxylic acid (2,6 NDC, 99%) were dissolved in 10 mL of N,N-dimethylformamide (DMF, ReagentPlus®, ≥99%), by stirring using a magnetic stirrer (model HS 15 from Misung Scientific Co., Ltd., Korea) at a speed of 5 rps for 5 min. Then, the solution was poured in a 23 mL Teflon autoclave bomb calorimeter (model 4781 microwave digestion bomb from Parr Instrument Company, USA) and heated in a microwave oven (model RCMT5088 W from Frigidaire Company, USA) at 160 W for 5 min. To separate the produced particles (Fe-NDC-M), the resulting mixture was centrifuged (centurion scientific centrifuge, model EB Series, UK) at 5,500 rpm for 30 min. After that, the supernatant was removed and the pale yellow particles were washed twice with 5 mL of DMF to remove the unreacted materials. Finally, the collected MOF particles were dried in an electrical oven (Incubator Classic Line with natural convection model BD 23 from BINDER GmbH Company, Germany) at 100° C. for 1.5 h to evaporate all of the DMF.

### Example 1-2: Synthesis of MOFs by Solvothermally Using an Electrical Oven

0.2307 mmol (93.2 mg) of iron (III) nitrate nonahydrate (Fe(NO₃)₃.9H₂O, ACS reagent, ≥98%) and 0.2307 mmol (49.9 mg) of 2,6 naphtalenedicarboxylic acid (2,6 NDC, 99%) were dissolved in 10 mL of N,N-dimethylformamide (DMF, ReagentPlus®, ≥99%), by stirring using a magnetic stirrer (model HS 15 from Misung Scientific Co., Ltd., Korea) at a speed of 5 rps for 5 min. Then, the solution was heated in the electrical oven (Incubator Classic Line with natural convection model BD 23 from BINDER GmbH Company, Germany) at 100° C. for 24 hours. To separate the produced particles (Fe-NDC-O), the resulting mixture was centrifuged (centurion scientific centrifuge, model EB Series, UK) at 5,500 rpm for 30 min. After that, the supernatant was removed and the pale yellow particles were washed twice with 5 mL of DMF to remove the unreacted materials. Finally, the collected MOF particles were dried in an electrical oven (Incubator Classic Line with natural convection model BD 23 from BINDER GmbH Company, Germany) at 100° C. for 1.5 h to evaporate all of the DMF.

### Example 2: Characterization of MOFs

The resulting MOFs of Examples 1-1 and 1-2 were characterized using X-ray diffraction (XRD), scanning electron microscopy (SEM), energy-dispersive X-ray (EDS), Fourier transform infrared spectroscopy (FTIR), and thermogravimetric analysis (TGA) to determine their morphological features.

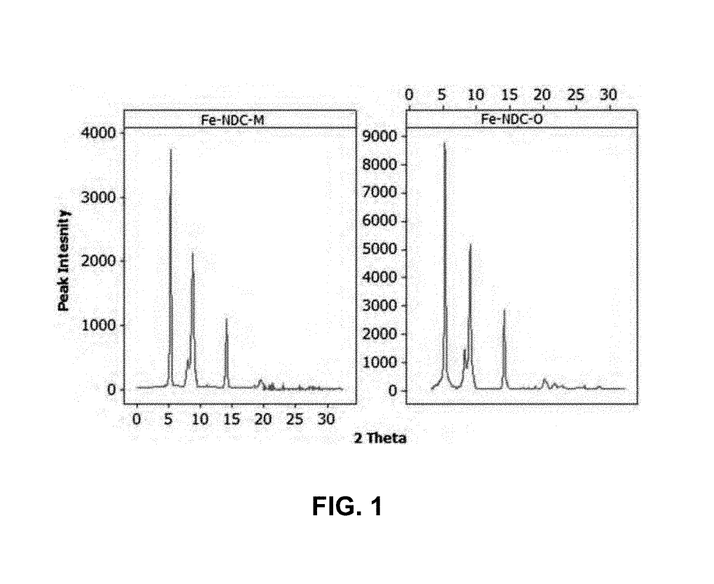

The XRD results were recorded from a conventional high resolution Bruker D8 Advance diffractometer (University of Sharjah) using a Cu Ka (k=1.54 Å) radiation source on silicon wafer from 3 to 40° (20) with a step size of 0.02° and 1 s (per step) in a continuous mode. The XRD results of Fe-NDC-M and Fe-NDC-O are illustrated in FIG. 1. The sharp peaks indicate that the synthesized MOFs had crystalline structures. On the other hand, the angles (20) of the sharp diffraction peak for the two MOFs are similar, which means that their crystal sizes are in the same or similar range.

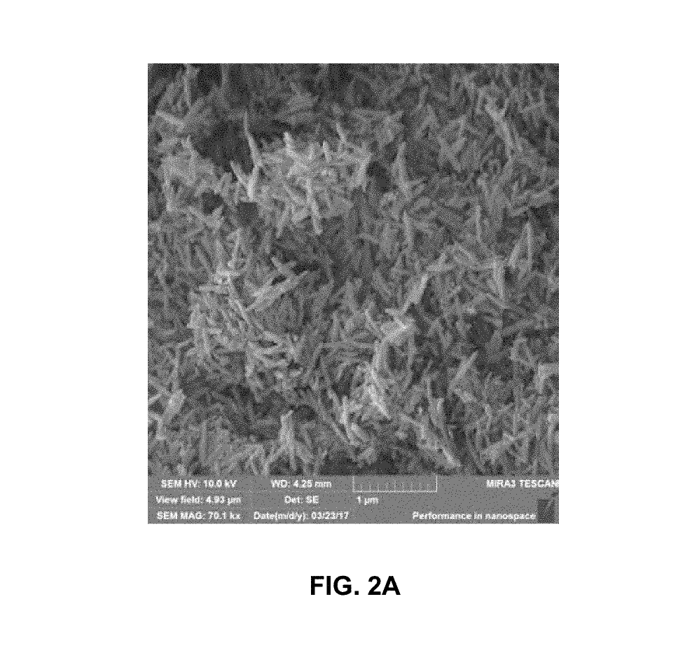

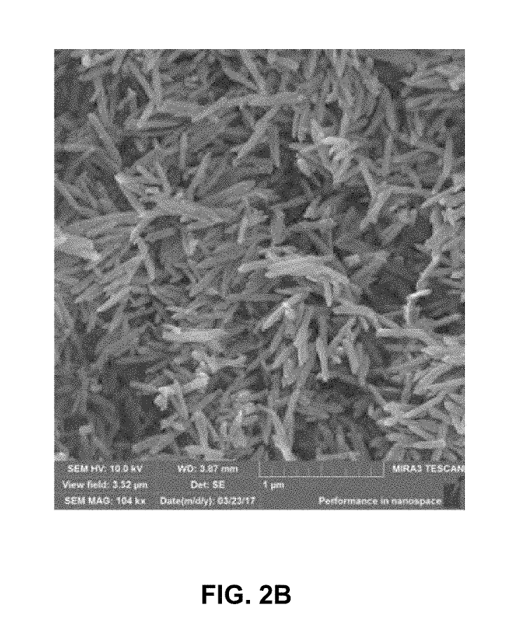

In order to get the SEM image of Fe-NDC-M, the sample was coated with gold in a SC7620 mini sputter coater with a carbon fiber evaporation attachment (Quorum Technologies Ltd., UK) to make it electrically conductive and then a scanning electron microscope (model VEGA III LMU with Oxford X-Act EDS, Tescan Orsay Holding Company, Czech Republic) was used. For Fe-NDC-O, another model (VEGA III XMU with Oxford X-Max 50 EDS) from the same company was used (University of Sharjah). Before starting the analysis, samples were coated with carbon using the same coater mentioned above. FIG. 2A illustrates the SEM image of Fe-NDC-M and FIG. 2B illustrates the SEM image of Fe-NDC-O. As illustrated in FIGS. 2A-B, most of the particles of Fe-NDC-M and Fe-NDC-O had a rod-like shape with a size in the nano range, with a diameter ranging between 50-80 nm and a length of 300-450 nm.

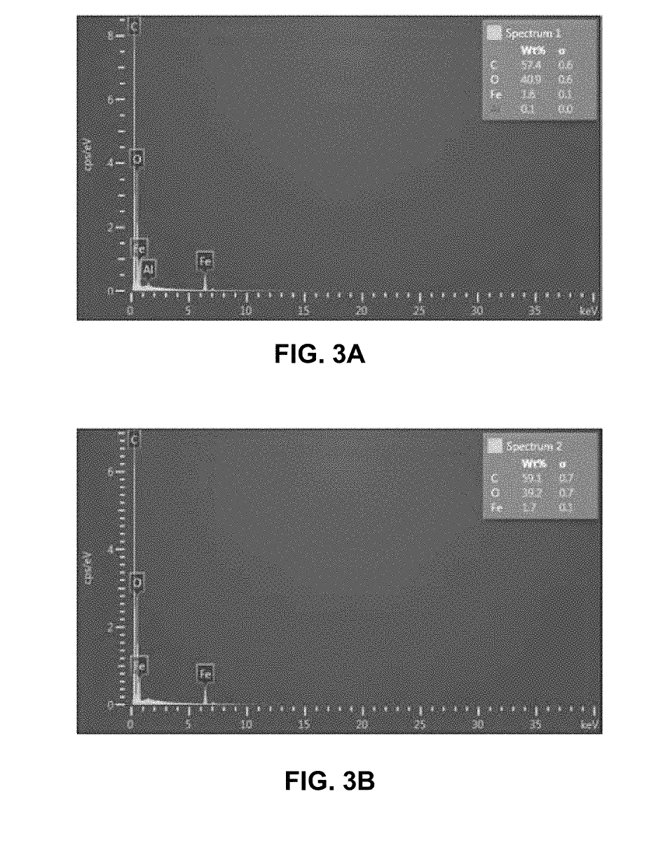

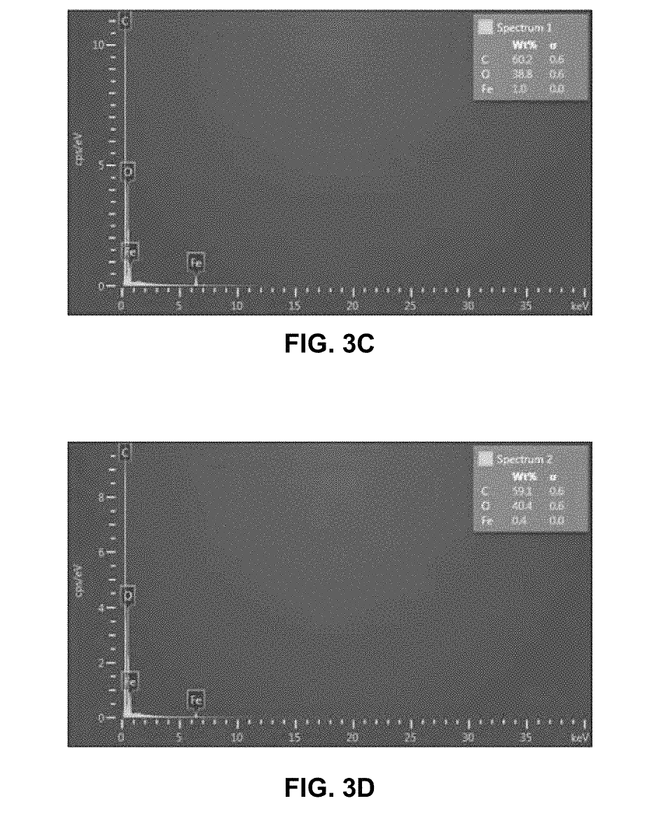

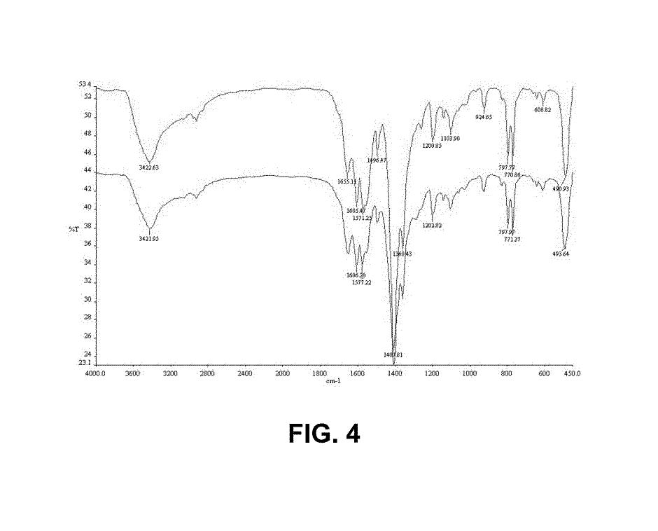

The Energy-dispersive X-ray (EDS) quantitative analysis for both MOFs was performed at two different positions of the sample as follows: a small amount of the sample was placed in an aluminum stub inside a scanning electron microscope (VEGA III XMU with Oxford X-Max 50 EDS) and the results were collected using the Aztec software. FIGS. 3A-B illustrate results of EDS analysis of Fe-NDC-M at the first and second positions, and FIGS. 3C-D illustrate results of EDS analysis of Fe-NDC-O at the first and second positions. As shown in FIG. 3A, EDS analysis of Fe-NDC-M at the first position yield the result of 57.4 wt % of carbon (C), 40.9 wt % of oxygen (O), and 1.6 wt % of iron (Fe). As shown in FIG. 3B, EDS analysis of Fe-NDC-M at the second position yield the result of 59.1 wt % of carbon (C), 39.2 wt % of oxygen (O), and 1.7 wt % of iron (Fe). As shown in FIG. 3C, EDS analysis of Fe-NDC-O at the first position yield the result of 60.2 wt % of carbon (C), 38.8 wt % of oxygen (O), and 1.0 wt % of iron (Fe). As shown in FIG. 3D, EDS analysis of Fe-NDC-O at the second position yield the result of 59.1 wt % of carbon (C), 40.4 wt % of oxygen (O), and 0.4 wt % of iron (Fe). As shown in FIGS. 3A-D, Fe-NDC-M and Fe-NDC-O showed the substantially similar composition wt. % at both positions.

Spectrum one FT-IR spectrometer (Perkin Elmer Company, USA) was used to get the FTIR patterns. First, tiny amounts of the MOFs were added to 200 mg of potassium bromide (FT-IR grade, ≥99% trace metals basis from Sigma-Aldrich Company) and ground manually. Then, they were compressed by applying 3,000 tons uniaxial pressure and the resulting discs were placed inside the spectrometer to obtain the FTIR patterns. The results were recorded using spectra software. FIG. 4 illustrates the FTIR patterns of Fe-NDC-M (upper) and Fe-NDC-O (lower). As shown in FIG. 4, Fe-NDC-M and Fe-NCD-O showed substantially same FTIR patterns, accordingly, the stretching and bending of the covalent bonds in molecules were similar in Fe-NDC-M and Fe-NDC-O.

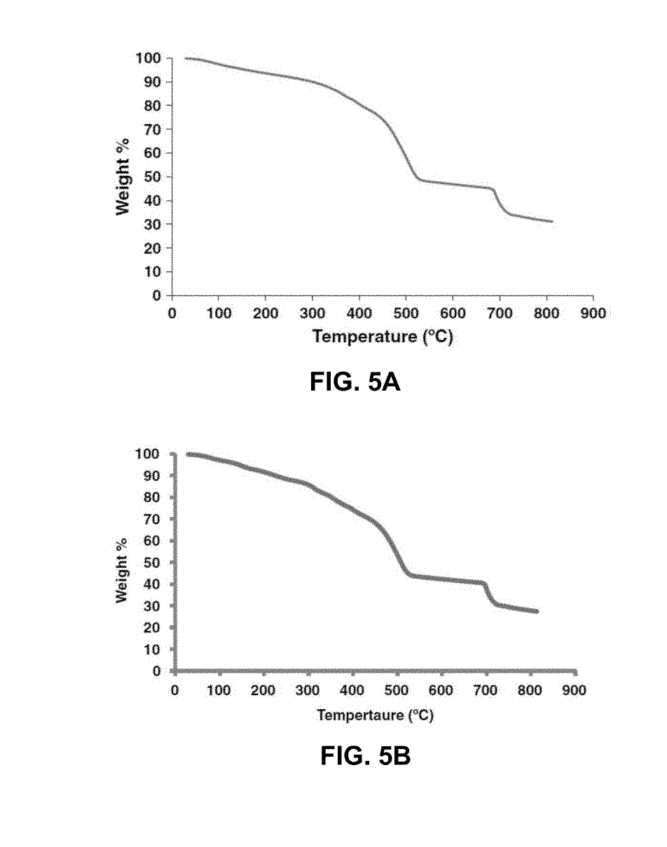

To test the thermal stability of the synthesized MOFs, the samples were analyzed using thermogravimetric analysis (TGA) Perkin Elmer TGA instrument at a temperature ramp rate of 15° C./min. FIGS. 5A-5B illustrate weight loss of Fe-NDC-M and Fe-NDC-O at increasing temperature. As shown in FIGS. 5A-5B, Fe-NDC-M exhibited a first significant decomposition between 300-522° C. and 300-507° C. for Fe-NDC-M and Fe-NDC-O, reaching to approximately 50 wt % of their initial mass. This may be assigned to the thermal degradation of the structurally coordinated 2,6-NDC ligands. The other weight loss was observed between 687-727° C. and 694-721° C. for Fe-NDC-M and Fe-NDC-O, respectively, where the remaining samples weights at the end of this step were 34 and 31 wt %. This loss may be assigned to the decomposition of the metal ion (Fe³⁺) from the MOF structure. As shown in FIGS. 5A-5B, difference between thermal stabilities of Fe-NDC-M and Fe-NDC-O were insignificant.

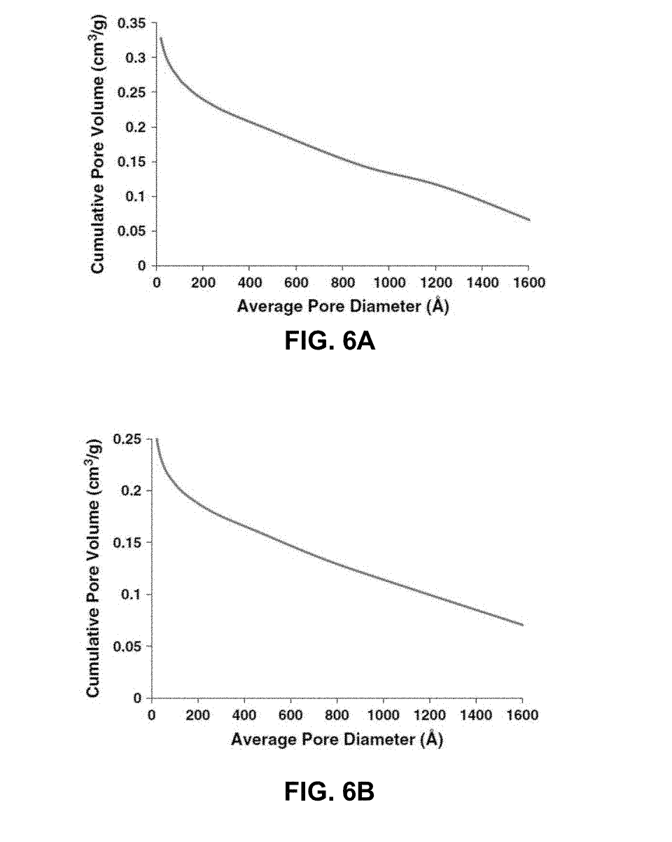

Nitrogen (N₂) absorption experiments were conducted for the analysis of volumes and sizes of the pores. The TriStar II 3020 micrometrics instruments were used and Joyner and Halenda (BJH) was applied for the quantitative evaluation of the pore size distribution. 0.0490 g Fe-NDC-M and 0.0321 g Fe-NDC-O were pre-dissolved at 250° C. for 18 h. Then, the samples were introduced into the N₂ gas sorption instrument at 77.35 K. FIGS. 6A-6B illustrate the BJH absorption cumulative pore volume of Fe-NDC-M and Fe-NDC-O, respectively. The BJH absorption average pore diameters were 148.551 \acute{Å} and 139.265 \ddot{Å} for the Fe-NDC-M and Fe-NDC-O, respectively.

### Example 3: Loading of Model Drug in MOFs

100 mg of calcein disodium salt (Sigma-Aldrich®), a model drug, were dissolved in 10 mL of neutral PBS to obtain a 15 mM solution. Some drops of HCl/NaOH were added carefully to maintain the pH at 7.4 (because it was observed that the pH values changed when calcein salt was added). Then, 130 mg of the dried MOFs from Examples 1-1 (Fe-NDC-M) and 1-2 (Fe-NDC-O) were stirred with the model drug solution for 48 h at 5 rps. The resulting mixture was centrifuged at 5,500 rpm for 30 min and the collected loaded particles were dried in an oven at 100° C. for 1 h.

To determine the loading efficiency (amount of model drug encapsulated/amount of model drug fed), the fluorescence values of: (1) 50 μL of 15 mM calcein disodium salt solution diluted in 3 mL of PBS, and (2) 50 μL of the supernatant (after the loading) diluted in 3 mL of PBS, were detected by a fluorescence spectroscopy (QuantaMaster QM 30, Photon Technology International, USA) connected to FelixGX software with the emission wavelength set between 490 and 515 nm. The solutions were diluted because of the self-quenching (decrease in the fluorescence intensity) of calcein at high concentrations. The loading experiments were repeated three times and the loading efficiencies were calculated using Equation (1).

$$Loading efficiency ( \% ) = \frac{{F}_{1} - {F}_{2}}{{F}_{1}} \times 100 \qquad ( 1 )$$

In Equation (1): F₁=the fluorescence of the model drug solution (before the loading); and F₂=the fluorescence of the supernatant after the loading.

Table 2 shows the loading efficiency of calcein disodium salt in Fe-NDC-M, and Table 3 shows the loading efficiency of calcein disodium salt in Fe-NDC-O. As shown inf Tables 2 and 3, both MOFs exhibited successful encapsulation of the model drug with loading efficiencies higher than 98%.

TABLE 2

| Run | F₁ | F₂ | Loading Efficiency (%) |
|---|---|---|---|
| 1 | 2.96 | 0.03 | 98.99 |
| 2 | 2.96 | 0.03 | 98.99 |
| 3 | 3.82 | 0.02 | 99.48 |

TABLE 3

| Run | F₁ | F₂ | Loading Efficiency (%) |
|---|---|---|---|
| 1 | 3.18 | 0.03 | 99.06 |
| 2 | 3.18 | 0.03 | 99.06 |
| 3 | 3.82 | 0.02 | 99.48 |

### Example 4: Release of the Model Drug

The release kinetics of the model drug from the MOF was studied. To determine the maximum release, one of the samples used in the release experiments without ultrasound at 37° C. and after exposure to 20 kHz ultrasound at room temperature were stirred at 1 rps for one week and then the fluorescence of 100 μL of the supernatant diluted in 2 ml of PBS was measured. These values were used to calculate the release percent for the experiments described above using Equation (2).

$$Drug release ( \% ) = \frac{{F}_{t} - {F}_{0}}{{F}_{ma x} - {F}_{0}} \times 100 \qquad ( 2 )$$

In Equation (2), Fₜ=the fluorescence at each time point. F₀=the fluorescence of the baseline (at t=0), and Fₘₐₓ=the fluorescence achieved after a week of stirring.

The power densities of the probe (at room temperature) and the sonication bath (at room temperature and 37° C.) were determined using a calibrated hydrophone (model 8103, Bruel and Kjaer Engineering Company, Denmark). Data of the acoustic spectrum (hydrophone responses, Vᵣₘₛ) were recorded using a digital storage oscilloscope (model TDS2002B, Tektronix Inc., USA). Then, equation (3) was used to calculate the power density. Table 4 illustrates power densities of a sonication bath (model DSC-50TH, Sonicor Inc., USA) and the probe.

$$\overline{I} = \frac{{\overline{V}}_{rm s}^{2} Q}{Z} \qquad ( 3 )$$

In Equation (3), 1=the average acoustic intensity, V̅ᵣₘₛ=the root-mean-squared voltage of the hydrophone signal, Q=the frequency-dependent calibration factor obtained from the manufacturer that relates pressure to voltage, Z=the acoustic impedance (Z for water=1.5×10⁶ kg/m²s).

TABLE 4

| Source of ultrasound and temperature | V̅ᵣₘₛ (V) | Power density (W/cm²) |
|---|---|---|
| Sonication bath at room temperature | 1.99 | 0.69 |
| Sonication bath at 37° C. | 1.86 | 0.61 |
| Probe at room temperature | 1.60 | 0.21 |

### Example 4-1: Release of the Model Drug from MOFs without Ultrasound

5 mL of PBS were added to 3 mg of the loaded MOFs. The fluorescence of the baseline (at time 0) was measured using the fluorescence spectroscopy after diluting 100 μL of the supernatant in 2 ml of fresh PBS. The remaining mixture was placed in a water bath at 37±0.5° C. The fluorescence values of 100 μL of the supernatant diluted in 2 ml of PBS were measured after 2, 4, 6, 8 and 10 min.

### Example 4-2: Release of the Model Drug from MOFs after 40 kHz Ultrasound

5 mL of PBS were added to 3 mg of the loaded MOFs. The fluorescence of the baseline (at time 0) was measured using the fluorescence spectroscopy after diluting 100 μL of the supernatant in 2 ml of fresh PBS. The remaining mixture was exposed to 40 kHz ultrasound using a sonication bath (model DSC-50TH, Sonicor Inc., USA) for 2 min at 37±0.5° C., at a power density of about 0.61 W/cm². The fluorescence values of 100 μL of the supernatant diluted in 2 ml of PBS were measured after 2, 4, 6, 8 and 10 min.

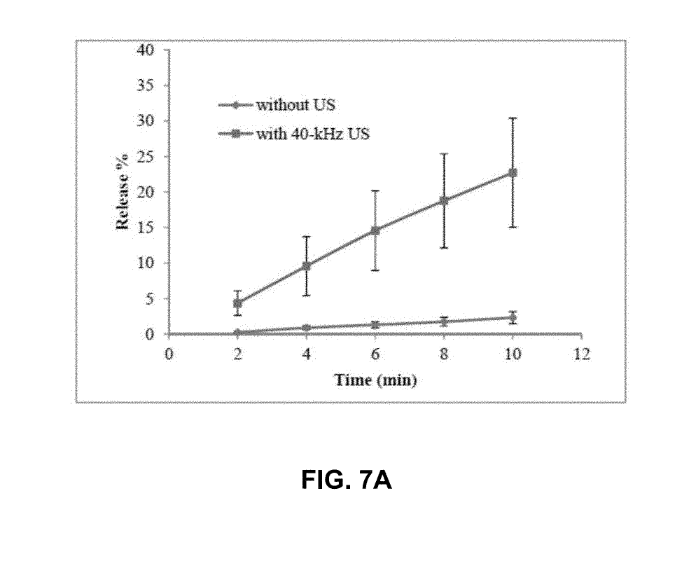

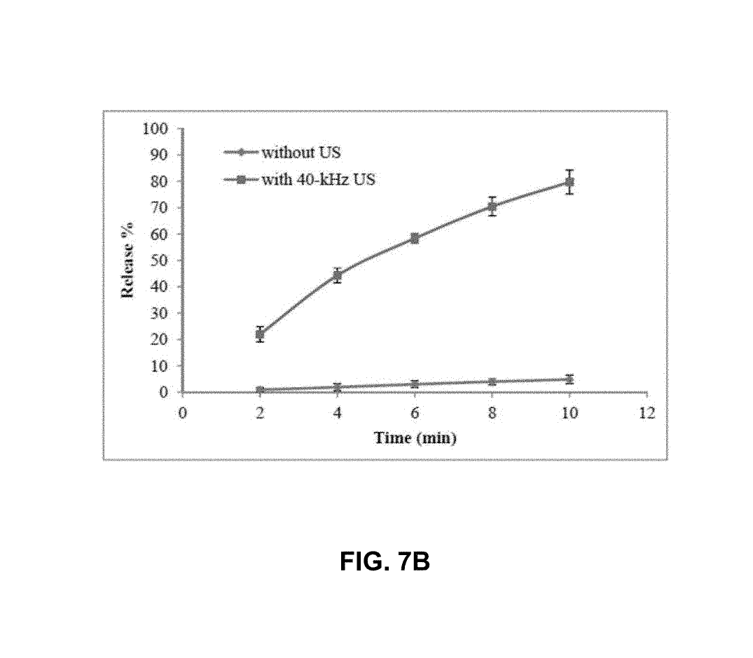

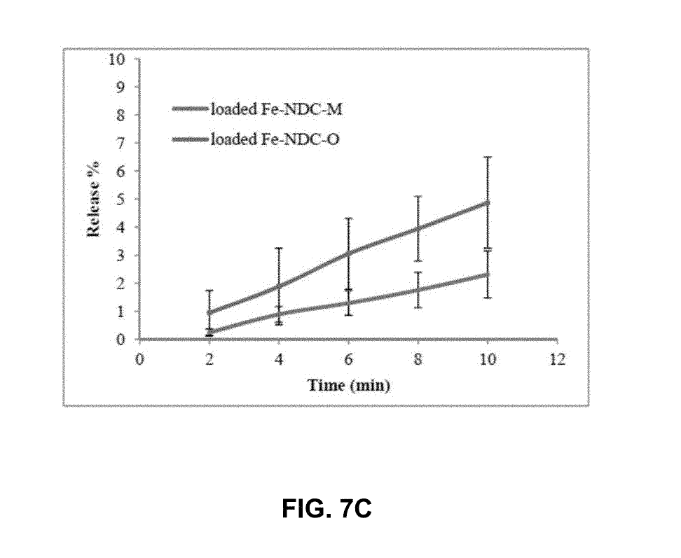

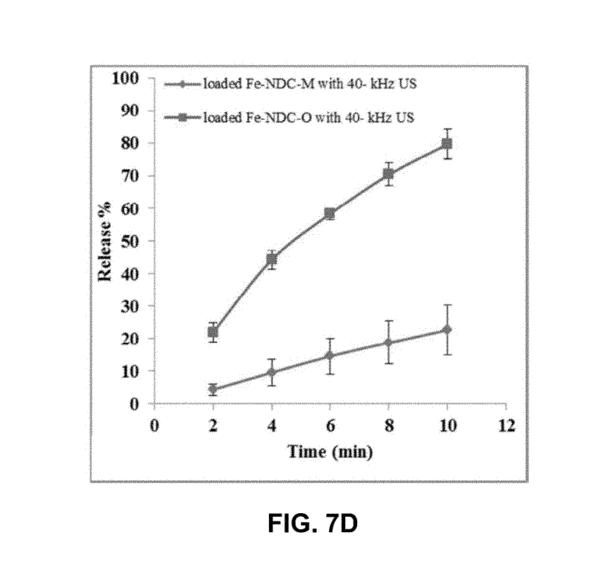

FIG. 7A illustrates the release kinetics of Fe-NDC-M of Examples 4.1 and 4.2. As shown in FIG. 7A, the average release percentages increased slightly without ultrasound to reach approximately 2.3% after 10 min, whereas it increased significantly after applying 40 kHz ultrasound and reached 22.7% after 10 min.

FIG. 7B illustrates the release kinetics of Fe-NDC-O of Examples 4.1 and 4.2. As shown in FIG. 7B, the average release percentages increased slightly without ultrasound to reach approximately 4.9% after 10 min, whereas it increased significantly after applying 40 kHz ultrasound and reached 79.7% after 10 min. This may confirm the role of ultrasonic waves in enhancing the oscillation of the nanoparticles which in turns enhances the drug release.

FIG. 7C illustrates a comparison of 1) the release profile of Fe-NDC-M MOFs without ultrasound (lower line) and 2) the release profile of Fe-NDC-O MOFs without ultrasound (upper line). As can be seen, without ultrasound, the average release percentage of calcein from the MOFs increases only slightly with time: neither MOF shows a significant release percentage after 10 minutes. Though, the release of calcein from Fe-NDC-O may be slightly higher than that from Fe-NDC-M.

FIG. 7D illustrates a comparison of 1) the release profile of Fe-NDC-O MOFs after application of about 40 kHz ultrasound at a power density of about 0.61 W/cm² (upper line) and 2) the release profile of Fe-NDC-M MOFs after application of about 40 kHz ultrasound at a power density of about 0.61 W/cm² (lower line). As can be seen, while both MOFs release calcein during ultrasonication faster than their respective controls, Fe-NDC-O MOFs release calcein during ultrasonication faster, e.g., significantly faster, than Fe-NDC-M MOFs in the same conditions. Fe-NDC-O MOFs experience a release percentage of about 80% after about 10 minutes whereas Fe-NDC-M MOFs experience a release percentage of about 23% in about the same time.

### Example 4-3: Release of the Model Drug from MOFs without Ultrasound and after Vortexing

5 mL of PBS were added to 3 mg of the loaded MOFs. The fluorescence of the baseline (at time 0) was measured using the fluorescence spectroscopy after diluting 100 μL of the supernatant in 2 ml of fresh PBS. The remaining mixture was placed in a water bath at 37±0.5° C. for 2 min. Then, it was vortexed at 1,000 rpm for 10 s using a vortexing unit (Heathrow Scientific® LLC, USA) and centrifuged at 5,500 rpm for 5 min. Then, the fluorescence values of 100 μL of the supernatant diluted in 2 ml of fresh PBS were measured. The remaining mixture was then vortexed at 1,000 rpm for 10 s. The same steps were repeated and the fluorescence values were measured after 2, 4, 6, 8 and 10 min.

### Example 4-4: Release of the Model Drug from MOFs after 40 kHz Ultrasound and Vortexing

5 mL of PBS were added to 3 mg of the loaded MOFs. The fluorescence of the baseline (at time 0) was measured using the fluorescence spectroscopy after diluting 100 μL of the supernatant in 2 ml of fresh PBS. The remaining mixture was exposed to 40 kHz ultrasound using the sonication bath (for 2 min at 37±0.5° C., at a power density of about 0.61 W/cm²). Then, it was vortexed at 1,000 rpm for 10 s using the vortexing unit and centrifuged at 5,500 rpm for 5 min. Then, the fluorescence values of 100 μL of the supernatant diluted in 2 ml of fresh PBS were measured. The remaining mixture was then vortexed at 1,000 rpm for 10 s. The same steps were repeated and the fluorescence values were measured after 2, 4, 6, 8 and 10 min.

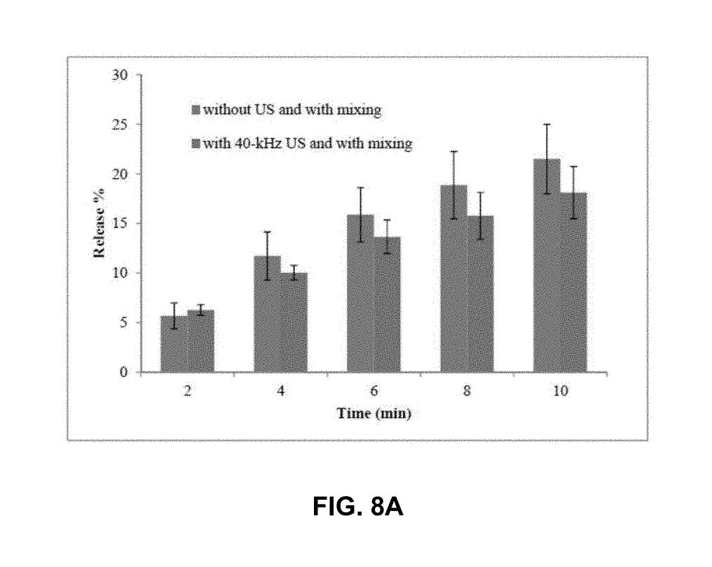

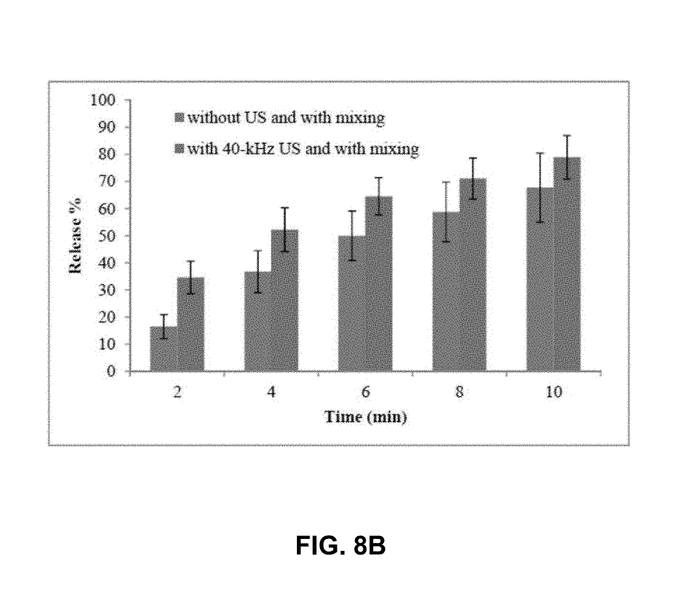

FIG. 8A illustrates the release kinetics of Fe-NDC-M of Examples 4.3 and 4.4. As shown in FIG. 8, the release percentages of both setups (with or without ultrasound) increased with time and reached 18.1% and 21.4% after 10 min. The average release percentages were found to be greater without ultrasound than after applying 40 kHz ultrasound. However, the difference was very small.

FIG. 8B illustrates the release kinetics of Fe-NDC-O of Examples 4.3 and 4.4. As shown in FIG. 8B, the release percentages of both setups (with or without ultrasound) increased with time and reached 78.9% and 67.7% after 10 min. The average release percentages were found to be greater without ultrasound than after applying 40 kHz ultrasound. However, the difference was very small.

### Example 4-5: Release of the Model Drug from MOFs (Fe-NDC-M) without Ultrasound

5 mL of PBS were added to 6 mg of the loaded MOFs (Fe-NDC-M). The fluorescence of the baseline (at time 0) was measured using the fluorescence spectroscopy after diluting 100 μL of the supernatant in 2 ml of fresh PBS. Then, the mixture was left at room temperature and the fluorescence values of 100 μL of the supernatant diluted in 2 ml of PBS were measured after 2, 4, 6, 8 and 10 min.

### Example 4-6: Release of the Model Drug from MOFs (Fe-NDC-M) after 20 kHz Ultrasound

5 mL of PBS were added to 6 mg of the loaded MOFs (Fe-NDC-M). The fluorescence of the baseline (at time 0) was measured using the fluorescence spectroscopy after diluting 100 μL of the supernatant in 2 ml of fresh PBS. Then, the sample was immersed in a 200 mL of distilled water bath and exposed to 20 kHz ultrasound (20 s on and 10 s off) using an ultrasonic probe (model CV334 with coupler model 630-0421, Sonics & Materials, Inc., USA) at a power density of about 0.21 W/cm². The fluorescence was measured for 100 μL of the supernatant diluted in 2 ml of PBS after 2, 4, 6, 8 and 10 min.

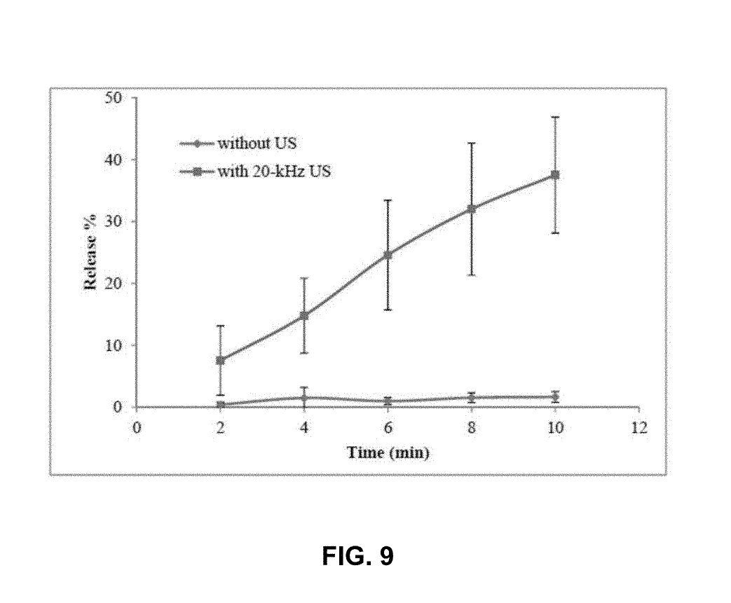

FIG. 9 illustrates the release kinetics of Fe-NDC-M of Examples 4.5 and 4.6. As shown in FIG. 9, the average release percentages increased slightly without ultrasound to reach approximately 1.7% after 10 min, whereas it increased significantly after applying 20 kHz ultrasound and reached 37.5% after 10 min.

### Example 5-1: Synthesis and Characterization of MOFs

The MOF (Fe-NDC-M) was synthesized using the procedure similar to the procedure described in Example 1-1, from 0.0932 g of iron nitrate nonahydrate, and 0.0499 g of 2,6 naphthalenedicarboxylic acid using 10 ml of N,N-dimethylformamide (DMF) as a solvent via microwave irradiation at 160 W for 5 min. The resultant particles were collected by centrifugation. Then, they were washed twice with DMF and dried in an oven at 100° C.

All chemicals required to prepare Fe-NDC-MOFs (iron nitrate nonahydrate (ACS reagent, ≥98%), 2,6-naphthalenedicarboxylic acid (95%), and N,N-dimethylformamide (DMF, ReagentPlus®, ≥99%)), methanol (puriss., meets analytical specification of Ph Eur, ≥99.7% (GC)), and MOF cytotoxicity chemicals (Roswell Park Memorial Institute (RPMI) medium, fetal bovine serum (FBS), penicillin-streptomycin, trypsin (0.25%), and propidium iodide) were purchased from Sigma-Aldrich company and used as received. Calcein disodium salt (abbreviated as cal. salt) was purchased from Honeywell Fluka™.

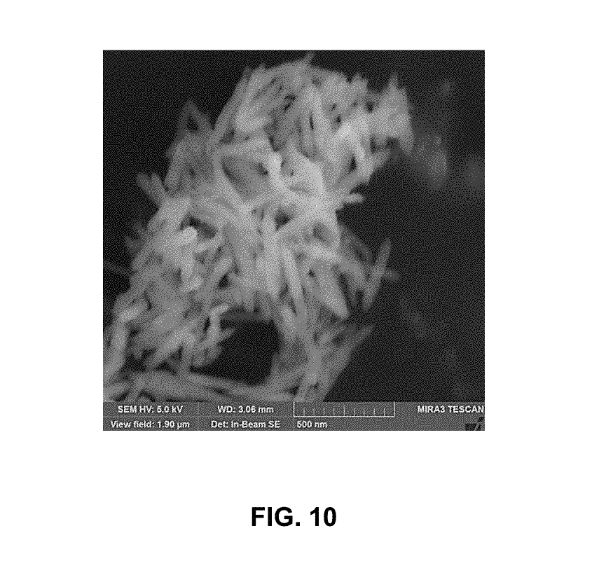

The structure and size of MOFs were determined from a scanning electron microscope (SEM) (MIRA3 XMU, Tescan Orsay Holding Company, Czech Republic). FIG. 10 illustrates the SEM image of the produced MOFs. The nanoparticles of MOFs had a rod-like shape with diameters of 50-80 nm and a length of 300-450 nm.

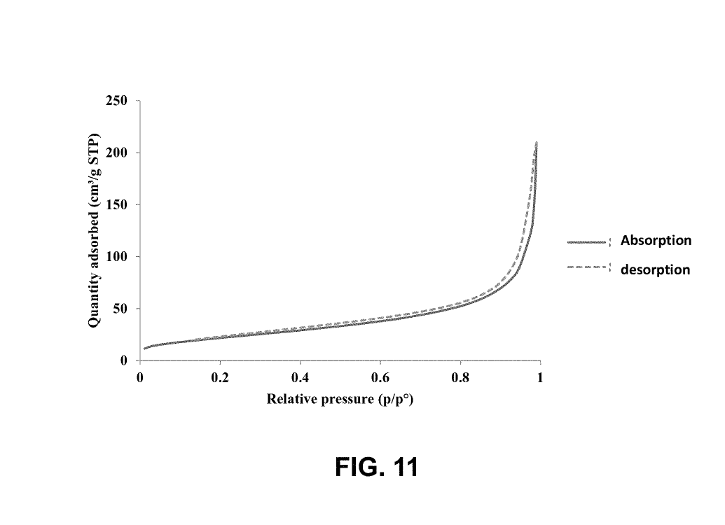

Also, the mechanism of pore filling was investigated via nitrogen gas physisorption isotherms using a TriStar II 3020 micromeritics instrument at 77.350 K (liquid nitrogen temperature). Volumetric adsorbed amounts of gas (cm³/g) were measured as a function of relativepressure (p/p°=absolute/saturationpressure). FIG. 11 illustrates a graph showing the absorbed amounts of gas measured as a function of relative pressure. FIG. 11 indicates physisorption isotherm type IV, characterizing the presence of mesopores which was confirmed by calculating the adsorption average pore width from Brunauer-Emmett-Teller (BET) theory to be ˜9.3 nm. This isotherm pattern is similar somewhat to the mesoporous TiO2, ZIF-8 @chitosan, and CSD (core-shell dual) @ZIF-8 composites.

### Example 5-2: Self-Quenching of Calcein Salt

Calcein is extensively used as a model drug to mimic the behavior of actual anticancer drugs because of its strong fluorescence that can be detected easily by ultraviolet-visible (UV) or fluorescence spectroscopes, as well as its low cost. Furthermore, the self-quenching fluorescence characteristic (decrease in the fluorescence intensity with increasing the concentration of calcein) at relatively high concentrations enables nanovesicles to load high amounts which can be released and observed upon subsequent dilution.

The self-quenching concentration of calcein salt was assessed to be considered while conducting the loading and release measurements. Several neutral solutions of the model drug in phosphate-buffered saline (PBS) were prepared with concentrations of: 0.0015, 0.0025, 0.0075, 0.005, 0.01, 0.015, 0.02, and 0.025 mM. The absorbance were measured using an Evolution™ 60S UV-visible spectrophotometer (Thermo Scientific™, USA) in the wavelength range between 400-550 nm. The average fluorescence intensities for the same solutions were measured in the range of 490-515 nm via a QuantaMaster™ fluorometer (Photon Technology International, USA). Then, the calibration curve was generated using the concentrations below the self-quenching.

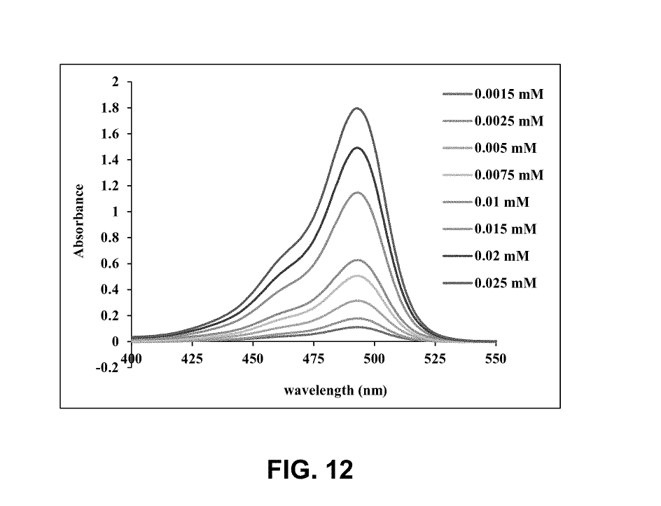

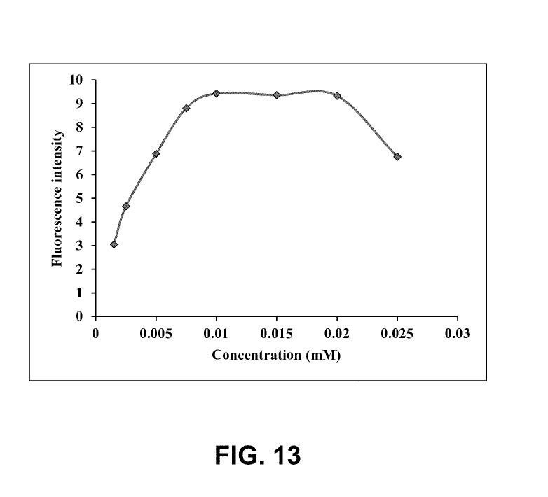

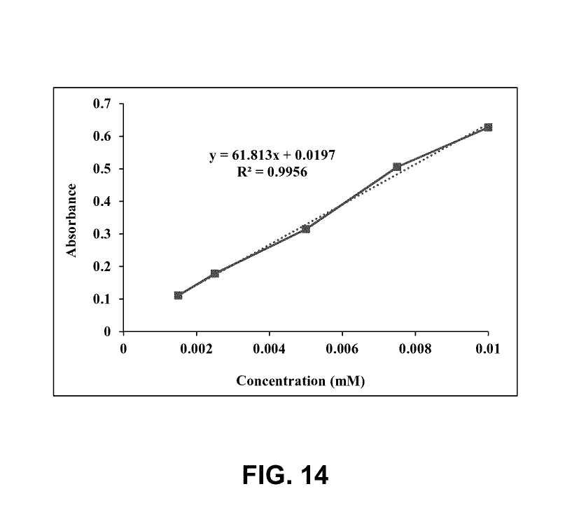

As illustrated in FIG. 12, the maximum absorbance of calcein salt was observed to exceed 1.0 at concentrations of 0.015 mM and above. On the other hand, as shown in FIG. 13, the average fluorescence intensity increased with increasing the concentration until 0.01 mM, and then it started to decrease. It was concluded that, the self-quenching concentration of calcein salt lies between 0.01-0.015 mM. Therefore, the calibration curve was generated using the first five concentrations as shown in FIG. 14.

### Example 5-3: Model Drug Loading

The loading experiments were performed via the simple impregnation method. First, the model drug was dissolved in methanol with NaOH to obtain a final concentration of 5 mg/ml. Then, 100 mg of Fe-NDC-M nanoparticles were soaked in the calcein salt solution at room temperature under magnetic stirring in the dark for two days. The resulting suspension was centrifuged and the supernatant was removed carefully. The collected loaded nanorods were dried to remove the solvent. All experiments were performed in triplicate. Although calcein was used for this particular experiment, in embodiments, the nanoparticles may be impregnated with any suitable drug, such as: doxorubicin, annamycin, daunorubicin, vincristine, cisplatin derivatives, paclitaxel 5-fluorouracil derivatives, camptothecin derivatives, and retinoids

To determine the amount of calcein salt loaded into the porous matrices after each experiment, aliquots from the supernatant and model drug stock solution were diluted in methanol (to avoid the self-quenching) and their absorbance peaks were measured using the UV-visible spectrophotometer. The characteristic peak of absorbance was read at a wavelength of 499 nm. Equations (4) and (5) were used to calculate the loading efficiencies and capacities, respectively.

$$\begin{matrix}Loading efficiency ( \% ) = \frac{{A}_{1} - {A}_{2}}{{A}_{1}} \times 100 & ( 4 ) \\ Loading capacity ( wt . \% ) = \frac{{m}_{loaded}}{{m}_{loaded} + {m}_{MOF}} \times 100 & ( 5 )\end{matrix}$$

In Equation (4), A₁=the absorbance of the model drug solution (before the loading) and A₂=the absorbance intensity of the supernatant after the loading. In Equation (5), m_{loaded}=the amount of loaded model drug (in mg), and m_{MOF}=the amount of empty MOF (in mg).

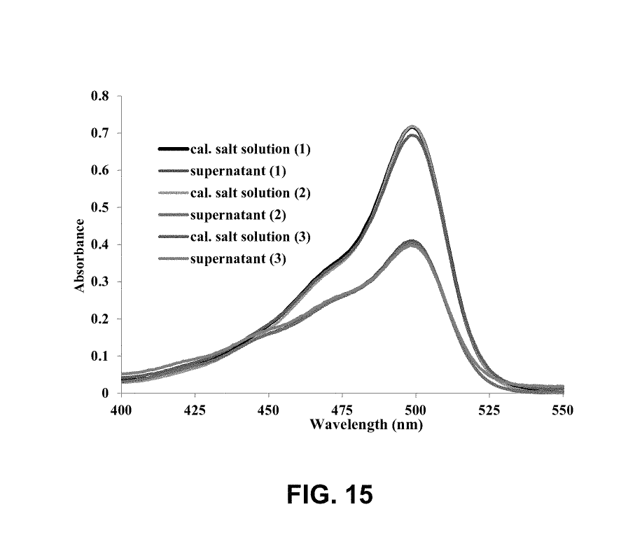

The concentration of loaded calcein onto Fe-NDC-M sample obtained from the UV-vis spectroscope is illustrated in FIG. 15. As shown in FIG. 15, the encapsulation efficiencies of calcein salt in the nano Fe-NDC-M for the first, second and third runs were 42.73, 43.77, and 42.91%, respectively, with an average of 43.13%. The loading capacity based on the average efficiency was calculated to be ˜17.74 wt. %.

### Example 5-4: Model Drug Release Kinetics

The release of calcein salt from the loaded Fe-NDC-M nanoparticles without ultrasound was studied. In addition, the effect of applying 40-kHz ultrasound with a power intensity of 1.0 W/cm² (using a sonication bath model DSC-50TH, Sonicor Inc., USA) on the model drug release was investigated for the first time on MOFs. All the experiments were carried out at 37° C. (to simulate the body temperature) for 3 mg of dried loaded MOFs in 3 ml of neutral PBS by measuring the absorbance after 2, 4, 6, 8, and 10 min. The release percentages were calculated using Equation (6).

$$CR \% = \frac{{A}_{t}}{{A}_{l}} \qquad ( 6 )$$

In Equation (6), CR=cumulative release, Aₜ=the absorbance at each time point, Aₗ=the absorbance of the loaded calcein salt.

Moreover, further trial was performed to confirm the effect of ultrasound in enhancing the release by keeping one the remaining samples that were used in the second run of the release experiments without and with US at 37° C. for an hour. Then, the absorbance was quantified by the UV-vis spectroscope.

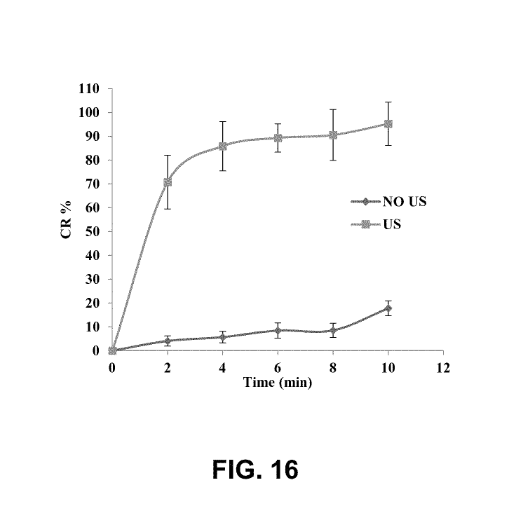

FIG. 16 illustrates release kinetics of calcein salt from Fe-NDC-M without ultrasound and with ultrasound. As shown in FIG. 16, the average amount of calcein salt released from the MOF increased slightly with time to reach ˜17.8% after 10 min without US. The release increased considerably when the ultrasound was applied to reach ˜95.2%. On the other hand, the samples left for an hour without any external stimuli did not show any further release. These promising results confirmed the role of the ultrasonic waves in enhancing the oscillation of the Fe-NDC-M nanorods which in turns triggers the drug release. Therefore, chemotherapeutic drugs loaded in MOFs can be delivered effectively upon applying the US at the tumor sites.

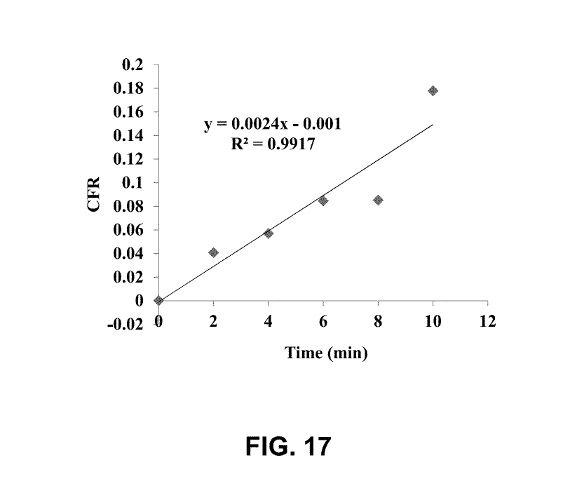

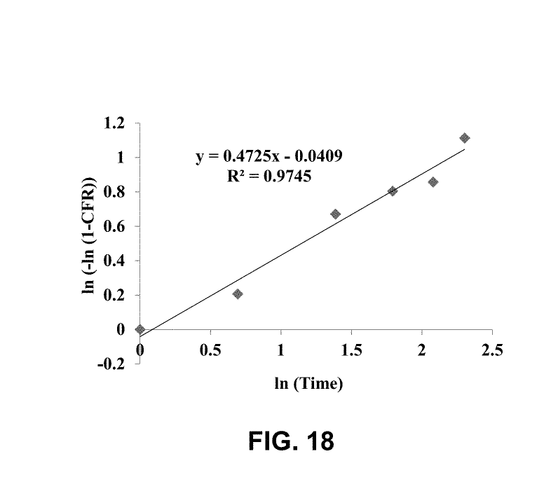

Further, the mechanism for the two release profiles (without and with ultrasound) were analyzed employing several mathematical models. In order to describe the drug delivery kinetics from the MOFs matrices, the release profile data were fitted to various mathematical models. The release profile without ultrasound was well-described by zero-order kinetics as shown in FIG. 17. The zero-order kinetics is represented by Equation (7). On the other hand, Weibull model described the release mechanism under the ultrasound, as shown in FIG. 18. The Weibull model is represented by Equation (8).

CFR=CFR₀+K₀t (7)

In Equation (7), CFR=cumulative fraction released at time (t), CFR₀=cumulative fraction released at time (0), K₀=the zero order release constant, t=time (in minutes).

$$ln ( - ln ( 1 - CFR ) ) = b ln ( t ) + ln ( \frac{1}{a} ) \qquad ( 8 )$$

In Equation (8), a=the scale parameter and b=the shape parameter.

### Example 5-5: Cytotoxicity of MOFs

The MCF-7 (ER-positive human breast adenocarcinoma) cells from ECACC (European collection of authentic cell culture) were utilized in the cell culture experiment. The cell lines were cultured in RPMI medium supplemented with 10% heat inactivated FBS and 1% penicillin-streptomycin. The cell cultures were maintained in T-75 flasks at 37° C. in a humidified atmosphere incubator with 5.0% C02 until cells were 80% confluent.

For the cytotoxicity tests, different amounts of as-synthesized Fe-NDC-M particles were dispersed in PBS via sonication to prepare solutions with concentrations of 12.5, 25, 50, 100, and 200 μg/ml. For the cytocompatibility analysis, the MCF-7 cells were harvested with trypsin and a cell density of 3×10⁵ cell/ml of growth medium was seeded in 6-well plates. The cells were incubated overnight allowing adherence to the wells.

Fresh media containing Fe-NDC-M solutions was introduced to the cells and incubated for 24 h. Then, the media were removed and the excess materials were washed by PBS. The cells were tripsinized and quantitative analysis of toxicity was conducted by direct cell counting using a flow cytometer (Beckman coulter FC500, CA, USA). Propidium iodide was used in the flow cytometry measurements to evaluate the % viability. All measurements were performed in triplicates of triplicates.

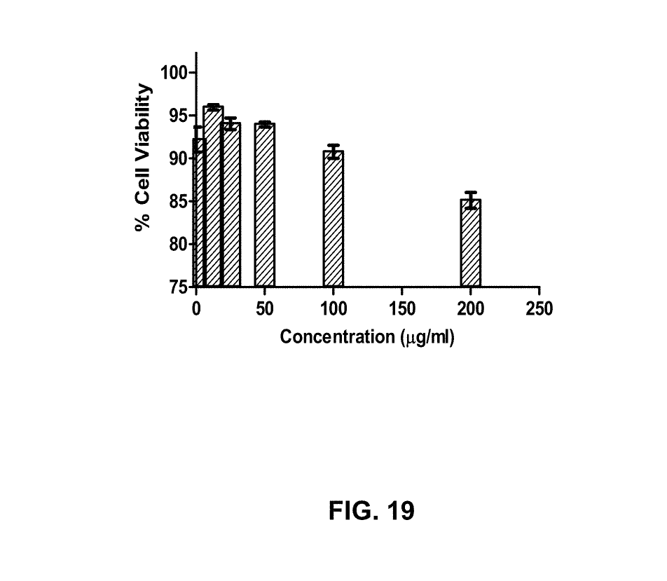

The effect of Fe-NDC-M toxicity on the cell viability is shown in FIG. 19. As seen, the tested MOF concentrations of 12.5, 25, 50, 100, and 200 μg/ml resulted in % viability of 95.95, 94.03, 93.95, 90.77, and 85.11, respectively. In addition, Graphpad Prism software was used to estimate the half maximal inhibitory concentration (IC50) of MOF towards MCF-7 cells, which was calculated to be 1022 μg/ml. From the investigation, it was concluded that Fe—NC-M seems to be very biocompatible with high % of viability even at relatively higher concentrations with IC50 comparable and moderate to other MOFs, indicating its potential use in various biomedical applications, especially for site-specific anticancer drug delivery.

### Terminology

The foregoing description and examples have been set forth merely to illustrate the disclosure and are not intended as being limiting. Each of the disclosed aspects and embodiments of the present disclosure may be considered individually or in combination with other aspects, embodiments, and variations of the disclosure. In addition, unless otherwise specified, none of the steps of the methods of the present disclosure are confined to any particular order of performance. Modifications of the disclosed embodiments incorporating the spirit and substance of the disclosure may occur to persons skilled in the art and such modifications are within the scope of the present disclosure. Furthermore, all references cited herein are incorporated by reference in their entirety.

Terms of orientation used herein, such as “top,” “bottom,” “horizontal,” “vertical,” “longitudinal,” “lateral,” and “end” are used in the context of the illustrated embodiment. However, the present disclosure should not be limited to the illustrated orientation. Indeed, other orientations are possible and are within the scope of this disclosure. Terms relating to circular shapes as used herein, such as diameter or radius, should be understood not to require perfect circular structures, but rather should be applied to any suitable structure with a cross-sectional region that can be measured from side-to-side. Terms relating to shapes generally, such as “circular” or “cylindrical” or “semi-circular” or “semi-cylindrical” or any related or similar terms, are not required to conform strictly to the mathematical definitions of circles or cylinders or other structures, but can encompass structures that are reasonably close approximations.

Conditional language used herein, such as, among others, “can,” “might,” “may,” “e.g.,” and the like, unless specifically stated otherwise, or otherwise understood within the context as used, is generally intended to convey that some embodiments include, while other embodiments do not include, certain features, elements, and/or states. Thus, such conditional language is not generally intended to imply that features, elements, blocks, and/or states are in any way required for one or more embodiments or that one or more embodiments necessarily include logic for deciding, with or without author input or prompting, whether these features, elements and/or states are included or are to be performed in any particular embodiment.

Conjunctive language, such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

The terms “approximately,” “about,” and “substantially” as used herein represent an amount close to the stated amount that still performs a desired function or achieves a desired result. For example, in some embodiments, as the context may dictate, the terms “approximately”, “about”, and “substantially” may refer to an amount that is within less than or equal to 10% of the stated amount. The term “generally” as used herein represents a value, amount, or characteristic that predominantly includes or tends toward a particular value, amount, or characteristic. As an example, in certain embodiments, as the context may dictate, the term “generally parallel” can refer to something that departs from exactly parallel by less than or equal to 20 degrees.

Unless otherwise explicitly stated, articles such as “a” or “an” should generally be interpreted to include one or more described items. Accordingly, phrases such as “a device configured to” are intended to include one or more recited devices. Such one or more recited devices can be collectively configured to carry out the stated recitations. For example, “a processor configured to carry out recitations A, B, and C” can include a first processor configured to carry out recitation A working in conjunction with a second processor configured to carry out recitations B and C.

The terms “comprising,” “including,” “having,” and the like are synonymous and are used inclusively, in an open-ended fashion, and do not exclude additional elements, features, acts, operations, and so forth. Likewise, the terms “some,” “certain,” and the like are synonymous and are used in an open-ended fashion. Also, the term “or” is used in its inclusive sense (and not in its exclusive sense) so that when used, for example, to connect a list of elements, the term “or” means one, some, or all of the elements in the list.

Overall, the language of the claims is to be interpreted broadly based on the language employed in the claims. The language of the claims is not to be limited to the non-exclusive embodiments and examples that are illustrated and described in this disclosure, or that are discussed during the prosecution of the application.

Although systems and methods for making and using MOFs, including control and targeted MOFs (including triggering MOFs using ultrasound), have been disclosed in the context of certain embodiments and examples, this disclosure extends beyond the specifically disclosed embodiments to other alternative embodiments and/or uses of the embodiments and certain modifications and equivalents thereof. Various features and aspects of the disclosed embodiments can be combined with or substituted for one another in order to form varying modes of systems and methods for making and using MOFs, including control and targeted MOFs (including triggering MOFs using ultrasound). The scope of this disclosure should not be limited by the particular disclosed embodiments described herein.

Certain features that are described in this disclosure in the context of separate implementations can be implemented in combination in a single implementation. Conversely, various features that are described in the context of a single implementation can be implemented in multiple implementations separately or in any suitable subcombination. Although features may be described herein as acting in certain combinations, one or more features from a claimed combination can, in some cases, be excised from the combination, and the combination may be claimed as any subcombination or variation of any subcombination.

While the methods and devices described herein may be susceptible to various modifications and alternative forms, specific examples thereof have been shown in the drawings and are herein described in detail. It should be understood, however, that the invention is not to be limited to the particular forms or methods disclosed, but, to the contrary, the invention is to cover all modifications, equivalents, and alternatives falling within the spirit and scope of the various embodiments described and the appended claims. Further, the disclosure herein of any particular feature, aspect, method, property, characteristic, quality, attribute, element, or the like in connection with an embodiment can be used in all other embodiments set forth herein. Any methods disclosed herein need not be performed in the order recited. Depending on the embodiment, one or more acts, events, or functions of any of the algorithms, methods, or processes described herein can be performed in a different sequence, can be added, merged, or left out altogether (e.g., not all described acts or events are necessary for the practice of the algorithm). In some embodiments, acts or events can be performed concurrently, e.g., through multi-threaded processing, interrupt processing, or multiple processors or processor cores or on other parallel architectures, rather than sequentially. Further, no element, feature, block, or step, or group of elements, features, blocks, or steps, are necessary or indispensable to each embodiment. Additionally, all possible combinations, subcombinations, and rearrangements of systems, methods, features, elements, modules, blocks, and so forth are within the scope of this disclosure. The use of sequential, or time-ordered language, such as “then,” “next,” “after,” “subsequently,” and the like, unless specifically stated otherwise, or otherwise understood within the context as used, is generally intended to facilitate the flow of the text and is not intended to limit the sequence of operations performed. Thus, some embodiments may be performed using the sequence of operations described herein, while other embodiments may be performed following a different sequence of operations.

Moreover, while operations may be depicted in the drawings or described in the specification in a particular order, such operations need not be performed in the particular order shown or in sequential order, and all operations need not be performed, to achieve the desirable results. Other operations that are not depicted or described can be incorporated in the example methods and processes. For example, one or more additional operations can be performed before, after, simultaneously, or between any of the described operations. Further, the operations may be rearranged or reordered in other implementations. Also, the separation of various system components in the implementations described herein should not be understood as requiring such separation in all implementations, and it should be understood that the described components and systems can generally be integrated together in a single product or packaged into multiple products. Additionally, other implementations are within the scope of this disclosure.

Some embodiments have been described in connection with the accompanying figures. Certain figures are drawn and/or shown to scale, but such scale should not be limiting, since dimensions and proportions other than what are shown are contemplated and are within the scope of the embodiments disclosed herein. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, any methods described herein may be practiced using any device suitable for performing the recited steps.

The methods disclosed herein may include certain actions taken by a practitioner; however, the methods can also include any third-party instruction of those actions, either expressly or by implication. For example, actions such as “positioning an electrode” include “instructing positioning of an electrode.”

Various embodiments and examples of systems and methods for making and using MOFs, including control and targeted MOFs (including triggering MOFs using ultrasound), are disclosed herein. Although the systems and methods for making and using MOFs, including control and targeted MOFs (including triggering MOFs using ultrasound), have been disclosed in the context of those embodiments and examples, this disclosure extends beyond the specifically disclosed embodiments to other alternative embodiments and/or other uses of the embodiments, as well as to certain modifications and equivalents thereof. This disclosure expressly contemplates that various features and aspects of the disclosed embodiments can be combined with, or substituted for, one another. Thus, the scope of this disclosure should not be limited by the particular disclosed embodiments described herein, but should be determined only by a fair reading of the claims that follow.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 1 V” includes “1 V.” Phrases preceded by a term such as “substantially” include the recited phrase and should be interpreted based on the circumstances (e.g., as much as reasonably possible under the circumstances). For example, “substantially perpendicular” includes “perpendicular.” Unless stated otherwise, all measurements are at standard conditions including temperature and pressure.

What is claimed is:

1. A method of treating cancer cells in a mammal, the method comprising:
providing a therapeutic composition, the therapeutic composition comprising a nanocarrier comprising a metal organic framework with therapeutic drug loaded therein, the metal organic framework configured to release the therapeutic drug upon exposure to ultrasound;
delivering the therapeutic composition to the mammal;
allowing the nanocarrier to circulate throughout a circulatory system of the mammal for a time sufficient to allow aggregation of a therapeutic quantity of the nanocarrier at a treatment area; and
applying ultrasound to the treatment area such that the nanocarrier releases the drug in the treatment area.

2. The method of claim 1, wherein the metal organic framework comprises iron (III) iron (Fe³⁺) and 2,6 naphthalenedicarboxylic acid.

3. The method of claim 1, wherein particles of the metal organic framework have a rod-like shape.

4. The method of claim 1, wherein particles of the metal organic framework have an average diameter between 50-100 nm.

5. The method of claim 1, wherein particles of the metal organic framework have an average length between 200-600 nm.

6. The method of claim 1, wherein the ultrasound applied to the treatment area comprises a low frequency ultrasound.

7. The method of claim 6, wherein the ultrasound applied to the treatment area has a frequency between 20 kHz and 40 kHz.

8. The method of claim 1, wherein the ultrasound applied to the treatment area has a power density of 0.1-2.0 W/cm².

9. The method of claim 1, wherein the ultrasound is applied with an ultrasound probe.

10. The method of claim 1, wherein the ultrasound is applied for 30 minutes or less.

11. The method of claim 1, wherein more than 20% of the loaded drug is released from the metal organic framework after applying the ultrasound.

12. The method of claim 1, wherein the drug is a chemotherapeutic drug.

13. The method of claim 12, wherein the drug is selected from the group consisting of doxorubicin, annamycin, daunorubicin, vincristine, cisplatin derivatives, paclitaxel 5-fluorouracil derivatives, camptothecin derivatives, and retinoids.

14. A therapeutic composition comprising:
a nanocarrier comprising a metal organic framework, wherein the metal organic framework comprises iron (III) iron (Fe³⁺) and 2,6 naphthalenedicarboxylic acid; and
a drug loaded at the metal organic framework.

15. The composition of claim 14, wherein particles of the metal organic framework have a rod-like shape.

16. The composition of claim 14, wherein particles the metal organic framework have an average diameter between 50-100 nm.

17. The composition of claim 14, wherein particles of the metal organic framework have an average length between 200-600 nm.
